(19) United States
(12) Patent Application Publication
(10) Pub. No.: US 2006/0200152 A1
(43) Pub. Date: Sep. 7, 2006

(54) Multi-purpose cutting tool

(76) Inventors: Ralph K. Karubian, Los Angeles, CA (US); John J. Leaman, Omaha, NE (US)

Correspondence Address: CHRISTIE, PARKER & HALE, LLP, PO BOX 7068, PASADENA, CA, 91109-7068, US

(21) Appl. No.: 11/064,336

(22) Filed: Feb. 23, 2005

(51) Int. Cl. A61B 17/14

(52) U.S. Cl. 606/82

(57) ABSTRACT

A multi-purpose cutting device for removing the spinal cord and spinal meninx lining of the spinal cord of a slaughtered animal which has been longitudinally cut in two along the length of the spinal cord. The cutting device comprises a rotary cutting tool having a hub with radially outwardly projecting cutting fingers equidistantly spaced apart around the hub. The cutting fingers have blunt ends and are of substantially uniform length. A pair of circular saw blades are rigidly secured to opposite faces of the cutting tool. The saw blades have a circumference that approximately matches the effective circumference of the working ends of the cutting fingers. In use, when the cutting device is powered by a drive motor, the cutting fingers and saw blades rotate in unison. The cutting fingers rotate to remove the spinal cord and spinal meninx from the carcass while the saw blades cut into bone along opposite sides of the meninx. The saw blades assist the cutting fingers in dislodging regions of bone material in which the spinal cord or meninx materials may be lodged.

### FIELD OF THE INVENTION

This invention relates to a multi-purpose cutting device, and more particularly, to a device used to remove the spinal cord and spinal meninx from a slaughtered animal.

### BACKGROUND

In the meat processing industry slaughtered animals such as cattle are longitudinally cut in two, after which the spinal cord is removed. In recent years this has become a more critical aspect of the process because of the spinal cord's possible contribution to “mad cow disease,” more specifically referred to as bovine spongiform encephalopathy (BSE).

USDA has required complete removal from certain cattle potentially infective tissues thought to be a carrier of BSE. These materials include the spinal cord and vertebral column of the carcass.

After the slaughtered animal is cut in two, a milling tool is typically used for removing the spinal cord and the spinal meninx lining the spinal cord of the animal. The meninx is a membrane that encloses the spinal cord, and it must be removed completely along with the spinal cord to comply with the USDA's requirements for addressing the BSE problem.

One prior art tool (described in German Patent No. 19729711) used for removing the spinal cord is available from the German manufacturer BVS Beratung Verkauf Service Grafische. This device comprises a rotary milling tool with radially extending fingers that scrape away the spinal cord material (the spinal cord and the meninx). The fingers on the cutting head are of fixed width. Since the spinal cord of an animal tends to vary in width along the length of the animal, tapering wider from tail to neck, the fixed width of the cutter does not effectively remove the entire spinal cord, including the entire spinal meninx along the entire length of the spinal column. Portions of the meninx lining tend to be lodged in the bone surrounding the spinal cord. The tool does not effectively remove bone since the cutting head is adapted more for scraping away the softer spinal cord and spinal meninx material.

In the process of cutting the carcass in two along the center of the spinal cord, the possibility exists that the longitudinal cut will not be perfectly on center. As often happens, the carcass is “missplit” in certain areas where the cut deviates from on center. This can cause “soft siding” where one side of the carcass has more bone than the other side, and in certain places, bone covers the entire spinal cord material. The prior art tool, which is unable to cut bone effectively, is unable to dislodge the bone of such a missplit animal, and is therefore unable to remove certain portions of the spinal cord and the surrounding meninx.

The present invention provides a cutting device that can remove the entire spinal cord and spinal meninx lining along the spinal cord of a slaughtered animal that has been split in two longitudinally along the spinal cord. The device can remove the entire spinal cord material (spinal cord and meninx) independently of changes in the width of the spinal cord along the length of the animal. The tool also can remove the entire spinal cord material independently of whether or not the carcass has been missplit along the spinal cord.

### SUMMARY OF THE INVENTION

Briefly, one embodiment of the invention comprises apparatus for removing the spinal cord and spinal meninx from a slaughtered animal. The apparatus comprises a motor driven rotary cutting head disposed in a housing which exposes an end portion of the cutting head. The cutting head includes a cutting tool having radially extending fingers with working ends disposed around the circumference of the cutting tool, and a pair of saw blades adjacent opposite sides of the cutting tool. The saw blades are rigidly affixed to the cutting tool to rotate in unison with it. The saw blades each have a working edge with a circumference effectively matching the circumference of the cutting tool. In use, the rotating fingers of the cutting tool can remove spinal cord and spinal meninx material while the rotary saw blades can remove bone along opposite sides of the spinal cord and meninx.

The cutting head of this invention is effective in removing the spinal cord and spinal meninx material completely from the slaughtered animal along the entire length of the spinal cord, independently of changes in its width from tail to neck of the animal. In addition, the saw blades are effective in removing bone, including bone material of a missplit carcass, so that the entire spinal cord and spinal meninx is removed also in those instances.

These and other aspects of the invention will be more fully understood by referring to the following detailed description and the accompanying drawings.

### BRIEF DESCRIPTION OF THE DRAWINGS

FIG. 1 is a perspective view showing a multi-purpose cutting device according to principles of this invention.

FIG. 2 is a side elevational view, partly in cross section, showing the relationship between the cutting fingers of the cutting tool and the rotary saw blades of the cutting head, the cross sectional view taken along line 2-2 of FIG. 3.

FIG. 3 is a cross sectional view taken along line 3-3 of FIG. 2.

FIG. 4 is an exploded perspective view showing components of the multi-purpose cutting device.

FIG. 5 is a cross sectional view showing the components of FIG. 4 in an assembled form.

FIG. 6 is a perspective view showing a cutting finger used on the cutting tool.

FIG. 7 is a cross sectional view showing the configuration of the cutting finger affixed to a rotary hub portion of the cutting tool.

FIG. 8 is a schematic view showing a spinal column bone, a spinal cord, and a meninx in cross section.

FIG. 9 is a schematic view similar to FIG. 8 showing an intended split line along a central or on-center axis of the spinal column.

FIG. 10 is a schematic view illustrating a missplit plane in relation to the spinal column and spinal meninx.

FIG. 11 is a schematic view showing the cutting tool of this invention in use exposing the meninx and spinal cord by cutting and removing bone on opposite sides of the spinal cord.

FIG. 12 is a schematic view showing the spinal column with spinal cord meninx and bone cover removed.

FIG. 13 is a side elevational view showing an optional embodiment of the cutting device.

### DETAILED DESCRIPTION

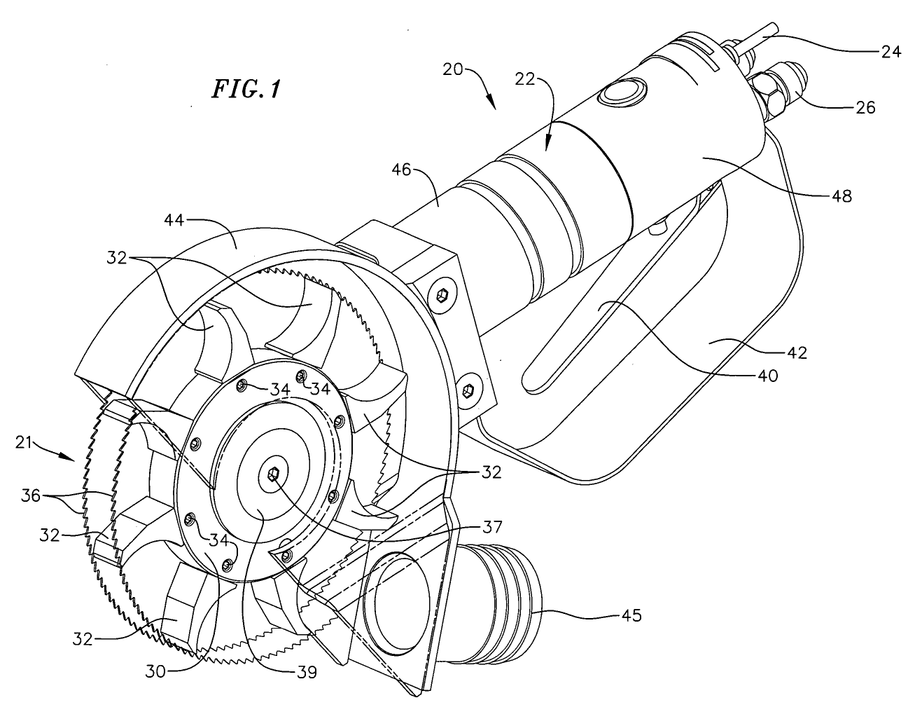

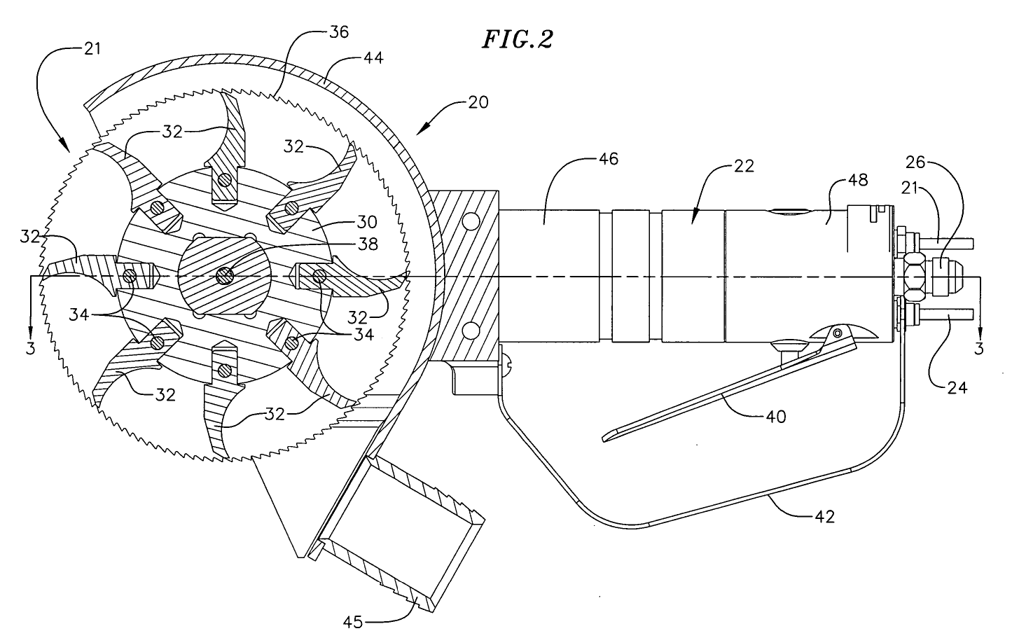

Referring to FIGS. 1 to 3, a multi-purpose cutting device 20 according to principles of this invention includes a cutting head 21 affixed to an elongated handle 22 which internally carries a hydraulic valve for operating a drive motor contained in the handle assembly, and an air valve providing controls for the hydraulic motor. FIG. 1 shows an air line connection 24 for compressed air leading to the pneumatic control valve, and a hydraulic hose connection 26 for a hydraulic line leading to the hydraulic valve.

The cutting head 21 at the opposite end of the handle includes a cylindrical hub 30 that supports a plurality of circumferentially spaced apart and radially extending cutting fingers 32. In the illustrated embodiment, the hub 30 carries eight equidistantly spaced apart fingers. The hub rotates in the counterclockwise direction with respect to the view shown in FIG. 1 when driven by the drive motor. The cutting fingers are affixed to an outer circumference of the hub by set screws 34. The cutting fingers are preferably of equal length, with their working ends extending to the same effective circumference around the rotating hub.

The cutting head further includes a pair of circular saw blades 36 rigidly affixed to opposite sides of the hub. The circular saw blades are aligned parallel to each other and rotate about a common axis 37 through the hub, along with the cutting fingers 32, under the power of the drive motor. A set screw 38 in the center of the hub rigidly affixes a flat, cylindrical blade holder 39 to the side of the hub. The saw blades have the same diameter, and the radius of each saw blade essentially matches the radius of the cutting fingers as measured from the axis of rotation through the hub. In a preferred embodiment, the cutting edges of the saw blades are spaced forward of the cutting ends of the cutting fingers by a small fraction of an inch. (The arrangement is described in more detail below.)

FIGS. 1 through 3 also shows other components of the cutting tool which include a hand-operated lever 40 attached to the handle, a guard 42 extending around the lever, and a partly circular guard 44 extending around the outer circumference of the cutting head and affixed to the handle portion of the tool. A vacuum connection 45 on a lower portion of the guard 44 connects to a vacuum source for removing material cut by the cutting head.

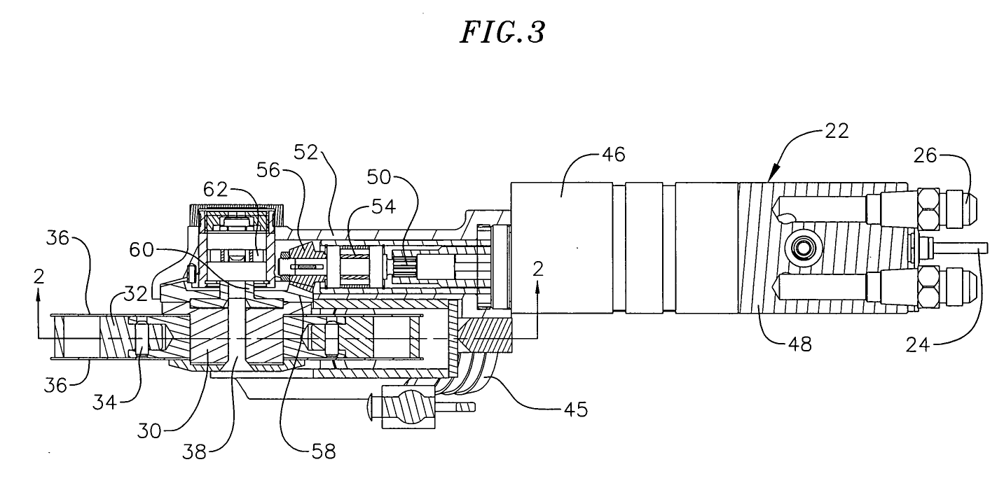

FIG. 3 illustrates the drive mechanism of the cutting head which includes a hydraulic drive motor 46 carried on the handle along with a valve body 48 for the pneumatic and hydraulic controls of the motor. A drive shaft 50 contained in a gear housing 52 is driven by the motor. The drive shaft is stabilized by a bearing 54 in the gear housing. The drive shaft is coupled to a pinion gear 56 which engages a bevel gear 58 centered on a mandrel 60. The hub 30 rotates on the mandrel 60. A bearing 62 contained in the gear housing stabilizes the mandrel.

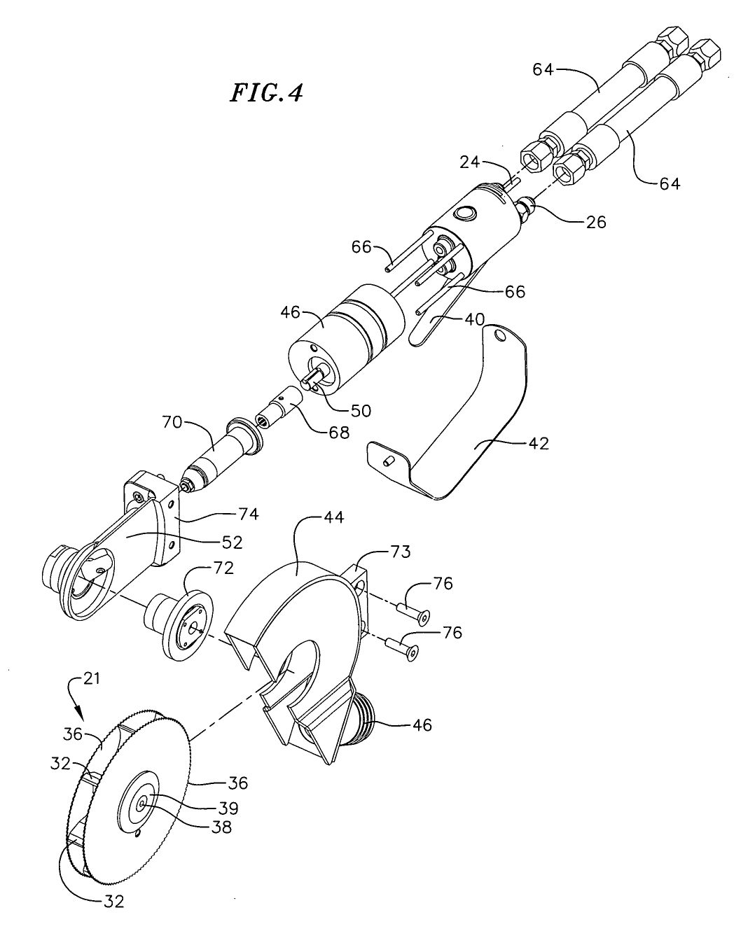

FIG. 4 illustrates an exploded assembly view of the cutting device. Hydraulic hoses 64 are coupled to the hydraulic hose connections 26 for supplying hydraulic fluid to the drive motor 46. The air line connections 24 supply air under pressure through air control lines 66 carried on the valve assembly 48. The valve assembly is coupled to the body of the drive motor 46. The drive shaft 50 from the body of the motor engages a coupling 68 which is aligned inside a drive cartridge 70 which contains the pinion gear 56. The pinion gear contained in the drive cartridge 70 engages the bevel gear 58 contained in the gear housing 52. The bevel gear 58 is contained in a drive gear cartridge 72 aligned with the axis of the cutting head 21. A plate 73 on the cutting head guard 44 is affixed to a base 74 of the gear housing 52 via set screws 76.

FIG. 5 is an assembly view showing components illustrated in the exploded view of FIG. 4. Briefly, the assembly view of FIG. 5 shows the hydraulic motor 46 and its valve assembly 48 in the handle portion 22 of the cutting device. This view also shows the gear housing 52 which contains the drive shaft 50 engaged with the drive motor via the coupling 68. This view also shows the drive shaft engaged with the pinion gear 56 which drives the bevel gear 58 and the mandrel 60. The drive assembly is shown adjacent to and connected to the hub 30 for powering the cutting head 21.

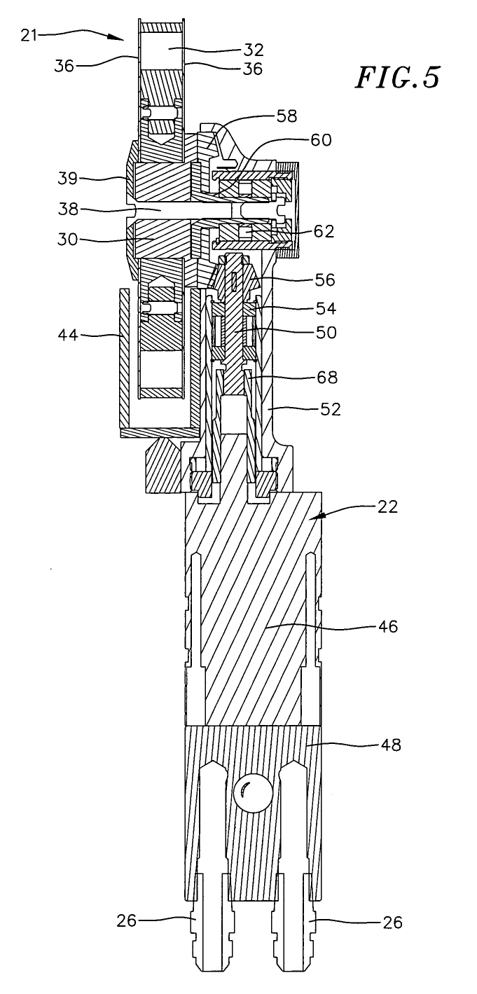

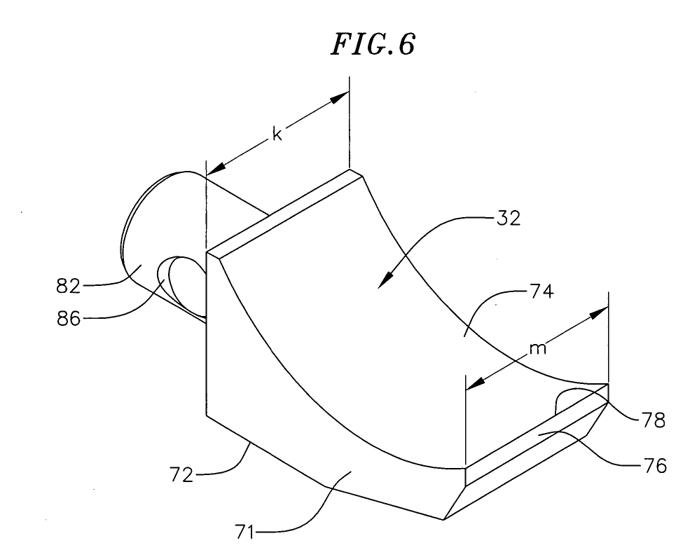

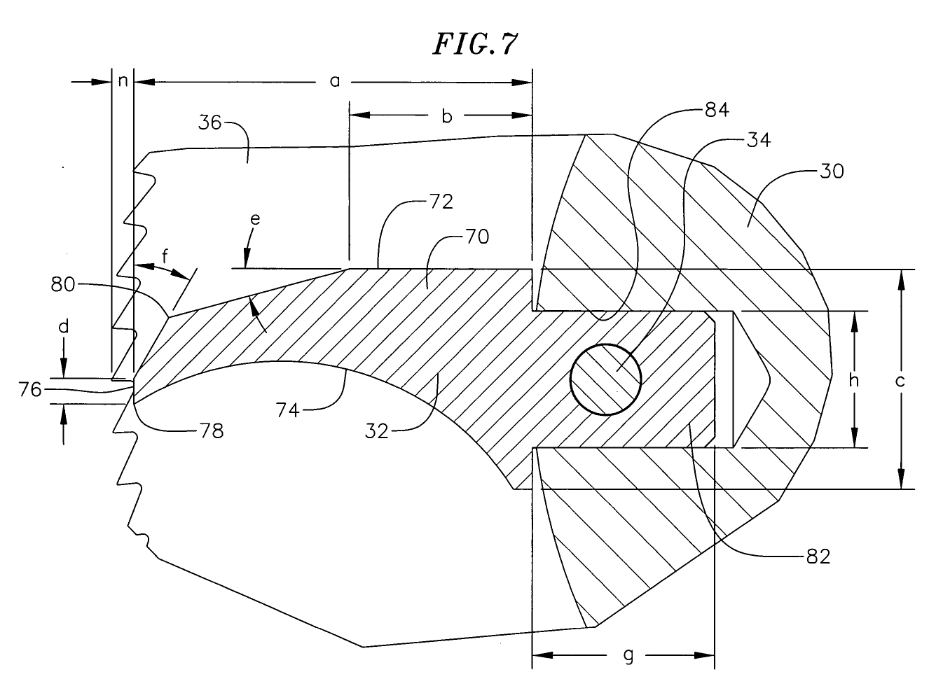

FIGS. 6 and 7 showed the detailed construction of the cutting fingers 32 and their relationship to the saw blades 36. The cutting fingers 32 each comprise and elongated body 70 made of solid metal. The body of the cutting finger has long flat parallel sides 71. The body of the finger tapers narrower toward its end and has an angular top surface 72 and a concavely curved bottom surface 74. A short flat cutting end 76 extends upwardly from an angular cutting edge 78. At the opposite end of the finger an angular edge 80 transitions between the top surface and an upper edge 80 of the flat cutting end 76.

The body of the finger narrows down to a cylindrically curved tubular shaft 82 which is seated in a corresponding passage 84 that faces radially outwardly from the hub. The set screws 34 extend through the hub and through corresponding passages 86 in each shaft for fastening each finger to the shaft so that the fingers extend radially outwardly from the hub.

Referring to FIG. 7, in one embodiment of the cutting head, the effective length a of the cutting finger is 1.090 inch, the length b of the flat surface 72 is 0.50 inch, the maximum outer dimension c of the body 70 is 0.60 inch, and the height d of the flat cutting edge 76 is 0.07 inch. The angled transition of the surface 72 shown at e is 15 degrees and the steeper angle f near the end of the cutting finger is 60 degrees. The shaft 82 has a length g of 0.50 inch and a diameter h of 0.374 inch. The maximum width k of the body 70 of the finger, as shown in FIG. 6, is 0.60 inch. As mentioned previously, the side walls 71 of the cutting fingers are parallel, and therefore, the cutting edge 76 also has a width m of 0.60 inch.

In the illustrated embodiment, the diameter of the saw blade is about 5.5 inches, and the diameter of the hub is about 3.3 inches.

As mentioned previously, the spacing n (FIG. 7) between the cutting edge of the saw blade and the flat cutting end of each finger is a fraction of an inch. In one embodiment, this spacing can range from about 0.05 to about 0.125 inch. In a preferred embodiment, the spacing is 0.06 inch.

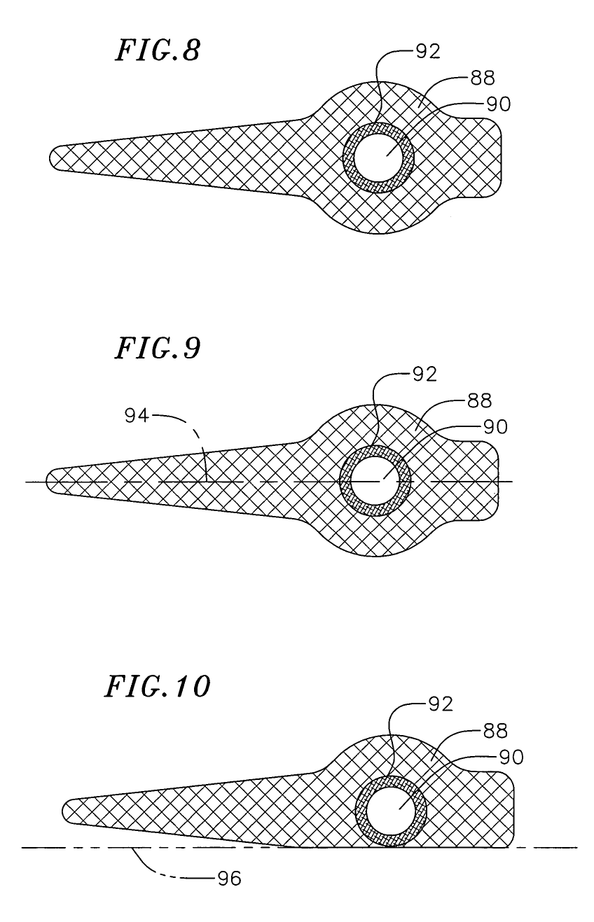

FIGS. 8, 9 and 10 illustrate a spinal column 88 of a slaughtered animal. The spinal column comprises bone and is shown in cross section to illustrate the spinal cord 90 which is contained in the bone and extends along the length of the animal. The spinal meninx 92 surrounds the spinal cord along the length of the spinal column. The axis 94 shown in FIG. 9 is an intended splitting axis and is shown on center when the spinal column is cut in two.

FIG. 10 illustrates a spinal column with a missplit plane 96 in which the spinal column is cut in two off center with respect to the intended splitting plane 94.

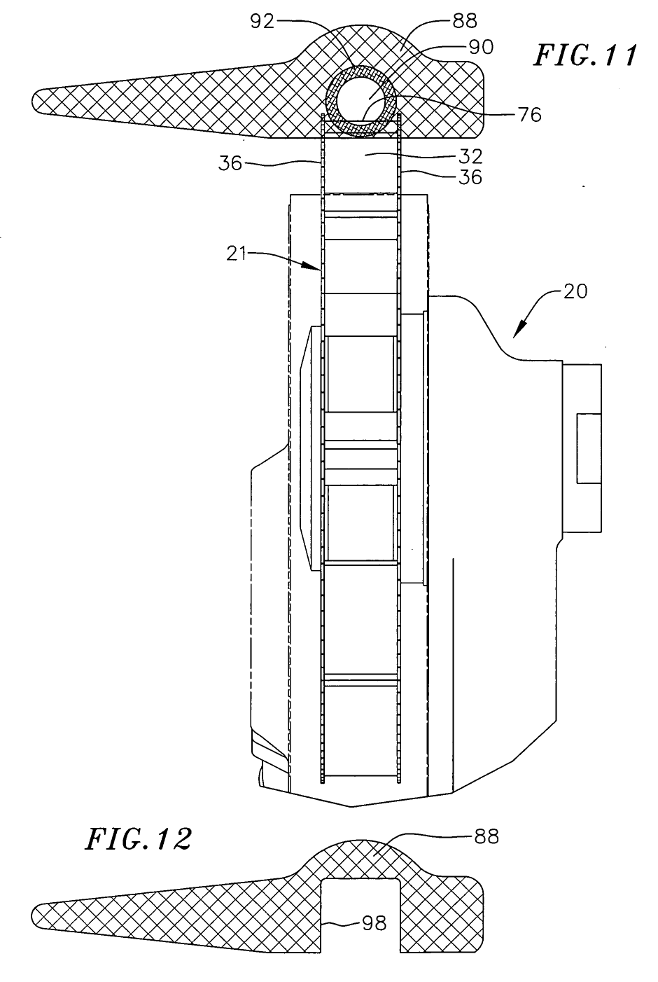

FIG. 11 shows the cutting tool of this invention exposing the meninx and spinal cord by cutting and removing bone contained in the region of the spinal column around the spinal cord and meninx.

The cutting fingers can remove the spinal cord and spinal meninx material along a spinal column which has been missplit as shown in the illustrated example of FIG. 11. The saw blades 36 are able to cut bone along opposite sides of the spinal cord to dislodge the bone so the rotary cutting fingers can remove the spinal material. The cutting head also is able to remove the entire spinal material via the saw blades cutting bone along both sides of the spinal cord and the spinal meninx, including dislodging bone to the rear side of the spinal cord to produce a clean cut 98 as shown in FIG. 12. The entire spinal cord and spinal meninx have been removed along with bone in this region surrounding the spinal material.

The width of the cutting fingers can essentially match the maximum width of the spinal cord and spinal meninx. With the saw blades able to remove bone simultaneously, the entire spinal material can be removed along the entire length of the spinal column independently of changes in the width of spinal column.

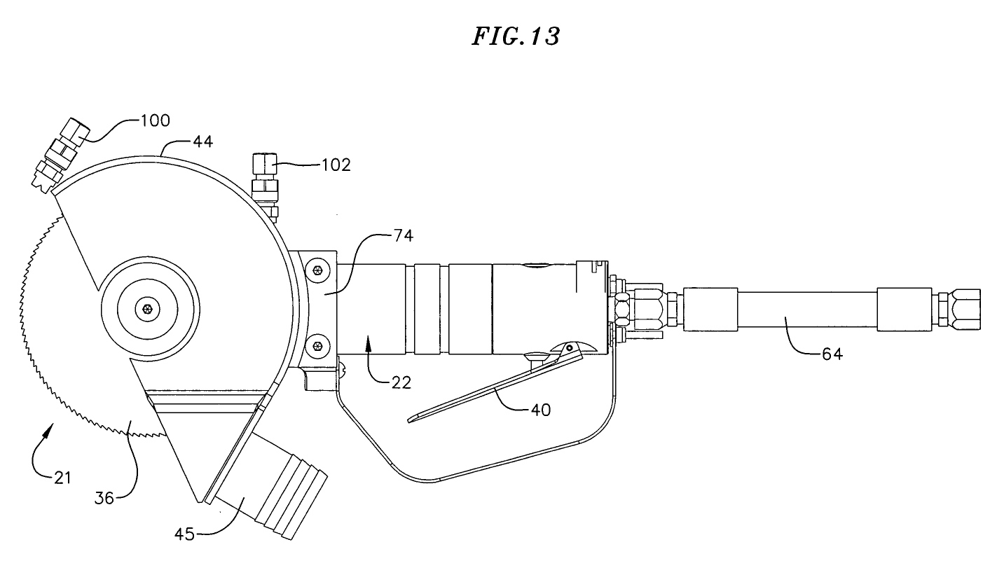

FIG. 13 illustrates an optional embodiment of the cutting tool which includes a spray nozzle 100 for hot water or steam or disinfectants such as lactic acid. The nozzle 100 is mounted on the guard 44 and positioned to spray the material downwardly toward the working edge of the cutting head 21. This embodiment further includes a spray nozzle 102 for hot water spray to sterilize the internal hub and blades. Valves (not shown) adjust the sprays. The valves can be positioned away from the tool.

What is claimed is:

1. Apparatus for removing the spinal cord and spinal meninx from a slaughtered animal, comprising a motor driven rotary cutting head disposed in a housing which exposes an end portion of the cutting head, the cutting head including a cutting tool having radially extending fingers with working ends disposed around a circumference of the cutting tool, and a pair of saw blades adjacent opposite sides of the cutting tool, the saw blades rigidly affixed to the cutting tool to rotate in unison therewith, the saw blades each having a working edge with a circumference effectively matching the circumference of the cutting tool, so that in use the fingers of the cutting tool can remove spinal cord and spinal meninx material while the rotating saw blades can remove bone along opposite sides of the cutting tool.

2. Apparatus according to claim 1 in which the fingers have blunt ends.

3. Apparatus according to claim 1 in which the saw blades are circular.

4. Apparatus according to claim 3 in which the saw blades are parallel and of uniform diameter.

5. Apparatus according to claim 1 in which the working edges of the saw blades project beyond the working ends of a plurality of the cutting fingers.
